(19) United States
(12) Patent Application Publication
(10) Pub. No.: US 2011/0086398 A1
(43) Pub. Date: Apr. 14, 2011

(54) CELLULAR PRODUCTION OF HYDROXYVALERATES FROM LEVULINATE

(76) Inventors: Collin Hunter Martin, North Wales, PA (US); Kristala Lanett Jones Prather, Cambridge, MA (US)

(21) Appl. No.: 12/935,378

(22) PCT Filed: Apr. 3, 2009
(86) PCT No.: PCT/US09/02132

§ 371 Date: Dec. 21, 2010

Related U.S. Application Data
(60) Provisional application No. 61/042,573, filed on Apr. 4, 2008.

(51) Int. Cl. C12P 7/42; C12N 1/21; C12N 5/10; C12N 1/15

(52) U.S. Cl. 435/146; 435/252.3; 435/252.34; 435/325; 435/254.11; 435/348

(57) ABSTRACT

The invention relates to cells that recombinantly express tesB and produce one or more hydroxyacids such as 3-hydroxyvalerate (3HV) and/or 4-hydroxyvalerate (4HV).

### RELATED APPLICATIONS

This application claims the benefit under 35 U.S.C. §119(e) of U.S. Provisional Application Ser. No. 61/042,573, entitled “Microbial Production of Hydroxyvalerates from Levulinate,” filed on Apr. 4, 2008, which is herein incorporated by reference in its entirety.

### GOVERNMENT INTEREST

This work was funded in part by the Synthetic Biology Engineering Research Center (SynBERC) funded by the National Science Foundation (Grant Number 0540879). The government has certain rights in this invention.

### Field of the Invention

The invention relates to the production of one or more hydroxyacids through recombinant gene expression.

### BACKGROUND OF THE INVENTION

Hydroxyacids are versatile, chiral compounds that contain both a carboxyl and a hydroxyl moiety, readily allowing for their modification into several useful derivatives (Lee, 2002; Chen, 2005). Specifically, hydroxyacids are used in the synthesis of antibiotics (Chiba, 1985), β- and γ-aminoacids and peptides (Park, 2001; Seebach, 2001), and as chiral synthetic building blocks (Lee, 2002). Hydroxyacids can also be used directly as nutritional supplements (Tasaki, 1999) and can be polymerized into biodegradable polyesters (polyhydroxyalkanoates, or PHAs) with interesting physical properties (Hazer, 2007).

Hydroxyacids are found in nature primarily polymerized as intracellular PHAs for energy storage in numerous organisms (Lenz, 2005). Of all the hydroxyacids, 3-hydroxybutyrate (3HB) is the most prolific, and several papers describe different means of producing monomeric 3HB (Lee, 1999; Gao, 2002; Liu, 2007). Longer chain length hydroxyacids, mainly 3-hydroxyvalerate (3HV), 4-hydroxyvalerate (4HV), 3-hydroxyhexanate and other medium chain length 3-hydroxyacids have been produced as constituents of various intracellular PHA co-polymers (Lee, 1999; Gorenflo, 2001; Park, 2002; Park, 2004). However, efficient production of these longer chain hydroxyacid monomers is complicated by issues such as low yields—typically less than 10% on a gram hydroxyacid per gram PHA basis for in vivo depolymerization from PHAs (Lee, 1999)—or the need for complicated chemical synthesis (Jaipuri, 2004) or purification (De Roo, 2002) procedures, most of which involve the use of large quantities of organic solvents.

Currently there exist three fundamental routes to the production of monomeric hydroxyacids: chemical synthesis, in vivo production of PHA polymers followed by depolymerization, and biological synthesis through non-PHA pathways. Chemical routes to hydroxyacid production are hampered by the high number of chemically reactive moieties in the hydroxyacid structure and the presence of a chiral center, and very few reports on their chemical synthesis are published (Jaipuri, 2004). There are, however, several reports on hydroxyacid production by depolymerizing PHAs through chemical or biological means (Lee, 2002 and references contained within). Chemical depolymerization of PHAs (Seebach, 1998) typically yields derivatives of hydroxyacids such as esters (De Roo, 2002). The subsequent chemical steps required to remove the chemical modifications from the hydroxyacids make this option for depolymerization unattractive. In vivo depolymerization can result in hydroxyacid dimer production (Lee, 1999). Both chemical and biological depolymerization methods require the production of a microbial PHA prior to depolymerization, which potentially complicates the process of hydroxyacid production. This additional step in the process may also result in poor product yields. Typical yields for PHA production are 0.3-0.5 gram PHA per gram carbon source, while typical yields for the recovery of hydroxyacids from depolymerized PHAs range from 6.7% to 87.5%, depending on the composition of the PHA and the depolymerization method employed (Wang, 1997; Lee 1999; Gorenflo, 2001; Ren, 2007).

Direct biological production of hydroxyacid monomers has been successfully demonstrated for 3HB, and titers of 2 g L⁻¹ and 12 g L⁻¹ on the shake flask and fed-batch scales have been reported (Gao, 2002). In these reports, 3-hydroxybutyrate is made from acetyl-CoA through the use of acetyl-CoA acetyltransferase (phbA), 3-hydroxybutyryl-CoA dehydrogenase (phbB), phosphotransbutyrylase (ptb), and butyrate kinase (buk) (Liu, 2000a; Liu 2000b; Gao, 2002). The last two of these enzymes were chosen to remove the CoA moiety from 3-hydroxybutyryl-CoA to yield free 3HB and were taken from Clostridium acetobutylicum, where they participate in the production of butyrate from butyryl-CoA. Recently, thioesterase II (tesB) from Escherichia coli K12 (Naggert, 1991) was successfully employed to directly hydrolyze the acyl-thioester of 3HB-CoA (Liu, 2007). While this pathway allows for the production of 3HB from glucose, it cannot be readily adapted for the production of higher chain length hydroxyacids or for hydroxyacids with different hydroxyl group positions.

### SUMMARY OF THE INVENTION

Described herein are methods for producing high titers of hydroxyacids from inexpensive and renewable carbon sources. A bioprocess has been developed for the production of hydroxyacids such as monomeric 3-hydroxyvalerate (3HV) and 4-hydroxyvalerate (4HV) from levulinic acid or a salt thereof (levulinate) in cells such as P. putida cells in culture. The bioprocess uses recombinant expression of tesB for removal of the CoA acyl carriers from intracellular hydroxyacids. Both minimal and rich media allow high-titer production of both 4HV and 3HV. This bioprocess represents a means for producing high chain length hydroxyacids from a feasible feedstock in the g L⁻¹ scale.

Aspects of the invention relate to a cell that produces one or more hydroxyacids, wherein the cell recombinantly expresses tesB. In some embodiments the one or more hydroxyacids, which are produced through conversion of levulinic acid to one or more hydroxyacids, comprises 3-hydroxyvalerate (3HV) and/or 4-hydroxyvalerate (4HV). The cell can be a bacterial cell, a fungal cell (including a yeast cell), a plant cell, an insect cell or an animal cell. In some embodiments the cell is a bacterial cell such as a Pseudomonas cell. In certain embodiments the cell is a Pseudomonas putida cell such as a Pseudomonas putida strain KT2440 cell. The recombinantly expressed tesB gene can be expressed from a plasmid or integrated into the genome of the cell. In some embodiments the tesB gene is a bacterial gene such as an E. coli gene.

The invention also includes methods for producing one or more hydroxyacids, the methods including culturing a cell that recombinantly expresses tesB, to produce one or more hydroxyacids and recovering the hydroxyacid from the cells. In some embodiments one or more of the hydroxyacids produced is 3-hydroxyvalerate (3HV), and the titer of 3-hydroxyvalerate (3HV) produced is at least 1 g L⁻¹ in minimal media, and at least 4 g L⁻¹ in rich media. In some embodiments one or more of the hydroxyacids produced is 4-hydroxyvalerate (4HV), and the titer of 4-hydroxyvalerate (4HV) produced is at least 1.5 g L⁻¹ in minimal media, and at least 9 g L⁻¹ in rich media.

The invention also includes methods for producing a cell that has increased hydroxyacid production. In some embodiments a cell that has increased hydroxyacid production recombinantly expresses tesB. The cell that has increased hydroxyacid production can be provided with levulinic acid which is converted into one or more hydroxyacids, such as 3-hydroxyvalerate (3HV) and/or 4-hydroxyvalerate (4HV). The cell that has increased hydroxyacid production can be a bacterial cell, a fungal cell (including a yeast cell), a plant cell, an insect cell or an animal cell. In some embodiments the cell is a bacterial cell such as a Pseudomonas cell. In certain embodiments the cell is a Pseudomonas putida cell such as a Pseudomonas putida KT2440 strain cell. According to methods of the invention the tesB gene can be expressed on a plasmid or integrated into the genome of the cell. In some embodiments the tesB gene is a bacterial gene such as an E. coli gene. The invention also includes methods for producing one or more hydroxyacids, the methods including producing a cell that has increased hydroxyacid production, culturing a population of said cells, and collecting one or more hydroxyacids from the population of cells that have increased hydroxyacid production. In some embodiments one or more of the hydroxyacids produced is 3-hydroxyvalerate (3HV), and the titer of 3-hydroxyvalerate (3HV) produced is at least 1 g L⁻¹ in minimal media, and at least 4 g L⁻¹ in rich media. In some embodiments one or more of the hydroxyacids produced is 4-hydroxyvalerate (4HV), and the titer of 4-hydroxyvalerate (4HV) produced is at least 1.5 g L⁻¹ on minimal media, and at least 9 g L⁻¹ on rich media.

The invention also includes a hydroxyacid produced by a cell culture wherein the cells within the cell culture have been genetically modified to recombinantly express tesB. In some embodiments the cells within the cell culture convert levulinic acid to one or more hydroxyacids such as 3-hydroxyvalerate (3HV) and/or 4-hydroxyvalerate (4HV). In some embodiments the hydroxyacid is produced in a cell that is a bacterial cell, a fungal cell (including a yeast cell), a plant cell, an insect cell or an animal cell. In some embodiments the cell is a bacterial cell such as a Pseudomonas cell. In certain embodiments the cell is a Pseudomonas putida cell such as a Pseudomonas putida KT2440 strain cell. The tesB gene can be expressed on a plasmid or integrated into the genome of the cell. In some embodiments the tesB gene is a bacterial gene such as an E. coli gene.

### BRIEF DESCRIPTION OF THE DRAWINGS

FIG. 1 is a graph representing a time-course of hydroxyvalerate accumulation in shake flask culture media by recombinant P. putida KT2440 harboring pRK415-tesB, grown at 30° C. in LB.

### DETAILED DESCRIPTION OF THE INVENTION

Aspects of the invention relate to methods and compositions for the production of one or more hydroxyacids through recombinant gene expression in cells. Described herein is the high titer production of hydroxyacids such as 4-hydroxyvalerate (4HV) and 3-hydroxyvalerate (3HV) from the carbon source levulinic acid in a cell that recombinantly expresses the gene tesB. This system represents an efficient new method for producing hydroxyacids, molecules that have a wide variety of applications.

According to aspects of the invention, cell(s) that recombinantly express tesB and the use of such cells in producing hydroxyacids are provided. It should be appreciated that the gene encoding thioesterase II (TesB) can be obtained from a variety of sources. In the embodiments discussed in the Example section presented herein, the TesB enzyme is encoded by a gene from E. coli. As one of ordinary skill in the art would be aware, homologous genes for this enzymes could be obtained from other species and could be identified by homology searches, for example through a protein BLAST search, available at the NCBI internet site (www.ncbi.nlm.nih.gov). A tesB gene can be PCR amplified from DNA from any source of DNA which contains this given gene. In some embodiments, the tesB gene is synthetic. Any means of obtaining a gene encoding TesB are compatible with the instant invention.

The invention encompasses any type of cell that recombinantly expresses tesB including prokaryotic and eukaryotic cells. In some embodiments the cell is a bacterial cell. In some embodiments the bacterial cell is a Pseudomonas cell such as a Pseudomonas putida (P. putida) cell. In certain embodiments the cell is a P. putida KT2440 cell. In other embodiments the cell is a fungal cell such as an S. cerevisiae cell. In other embodiments the cell is a mammalian cell or a plant cell. It should be appreciated that some cells compatible with the invention may express an endogenous copy of tesB as well as a recombinant copy.

In some embodiments, the gene encoding tesB is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of tesB for production of hydroxyacids is demonstrated in the Examples section using P. putida. The novel method for producing hydroxyacids can also be expressed in other bacterial cells, archael cells, fungi (including yeast cells), mammalian cells, plant cells, etc.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments tesB is expressed recombinantly in a bacterial cell. Bacterial cells according to the invention can be cultured in media of any type (rich or minimal) and any composition. Example 1 presents embodiments in which rich media (LB media) and minimal media (M9 media) were each found to be capable of producing 4HV and 3HV at the g L⁻¹ scale. As would be understood by one of ordinary skill in the art, routine optimization would allow for use of a variety of types of media. The selected medium can be supplemented with various additional components. Some non-limiting examples of supplemental components include glucose, antibiotics, IPTG for gene induction, and ATCC Trace Mineral Supplement. Similarly, other aspects of the medium, and growth conditions of the cells of the invention may be optimized through routine experimentation. For example, pH and temperature are non-limiting examples of factors which can be optimized. As presented in the Example section, factors such as choice of media, media supplements, and temperature can in some embodiments influence production levels of select hydroxyacids. In some embodiments manipulation of these factors influences the ratio of 3HV produced relative to 4HV. Thus in some embodiments these factors may be manipulated in order to produce more of a select hydroxyacid such as 3HV or 4HV relative to other hydroxyacids produced.

As presented in the Example section, it was discovered that temperature influences both the overall titer of hydroxyacids produced and in some embodiments the ratio of specific hydroxyacids produced such as the ratio of 4HV to 3HV produced. In some embodiments the temperature of the culture may be between 25 and 40 degrees. For example it may be 25, 26, 27, 28, 29, 30, 31, 32, 33, 34, 35, 36, 37, 38, 39 or 40 degrees, or any value in between. In certain embodiments the temperature is between 30 and 32 degrees including 30, 31 and 32 and any value in between. As would be understood by one of ordinary skill in the art, the optimal temperature in which to culture a cell for production of one or more hydroxyacids will be influenced by many factors including the type of cell, the growth media and the growth conditions.

Other non-limiting factors that can be varied through routine experimentation in order to optimize hydroxyacid production include the concentration and amount of levulinate provided, how often the media is supplemented with levulinate, and the amount of time that the media is cultured before harvesting the one or more hydroxyacids. In some embodiments levulinate may be added to the culture at t=0, 6, 12, 18, 24, 30, 36, 42, 45, 50, 55, 60, 65, 70, 75, 80, 85, 90, 95, 100, 110, 120, 130, 140, 150, 160 and/or greater than 160 hours, including all intermediate values. In some embodiments levulinate may be added to the culture 1, 2, 3, 4, 5, 6, 7, 8, 9, 10, 11, 12, 13, 14, 15 or more than 15 times. In some embodiments the cells may be cultured for 6, 12, 18, 24, 30, 36, 42, 45, 50, 55, 60, 65, 70, 75, 80, 85, 90, 95, 100, 110, 120, 130, 140, 150, 160 or greater than 160 hours, including all intermediate values. In some embodiments optimal production is achieved after culturing the cells for several days such as 3-4 days. However it should be appreciated that it would be routine experimentation to vary and optimize the above-mentioned parameters and other such similar parameters.

According to aspects of the invention, high titers of hydroxyacids are produced through the recombinant expression of tesB in a cell. As used herein “high titer” refers to a titer in the grams per liter (g L⁻¹) scale. The titer produced for a given hydroxyacid will be influenced by multiple factors including choice of media. In some embodiments the titer for production of 3-hydroxyvalerate (3HV) is at least 1 g L⁻¹ in minimal media. For example the titer may be 1, 2, 3, 4, 5, 6, 7, 8, 9, 10, or more than 10 g L⁻¹ including any intermediate values. In some embodiments the titer for production of 3-hydroxyvalerate (3HV) is at least 4 g L⁻¹ in rich media. For example the titer may be 4, 5, 6, 7, 8, 9, 10, 11, 12, 13, 14, or more than 14 g L⁻¹ including any intermediate values. In some embodiments the titer for production of 4-hydroxyvalerate (4HV) is at least 1.5 g L⁻¹ in minimal media. For example the titer may be 1.5, 2, 3, 4, 5, 6, 7, 8, 9, 10, or more than 10 g L⁻¹ including any intermediate values. In some embodiments the titer for production of 4-hydroxyvalerate (4HV) is at least 9 g L⁻¹ in rich media. For example the titer may be 9, 10, 11, 12, 13, 14, 15, 16, 17, 18, 19, 20, or more than 20 g L⁻¹ including any intermediate values.

The liquid cultures used to grow cells associated with the invention can be housed in any of the culture vessels known and used in the art. In some embodiments large scale production in an aerated reaction vessel such as a stirred tank reactor can be used to produce large quantities of the hydroxyacids associated with the invention.

Aspects of the invention include strategies to optimize hydroxyacid production from a cell. Optimized production of a hydroxyacid refers to producing a higher amount of a hydroxyacid following pursuit of an optimization strategy than would be achieved in the absence of such a strategy. One strategy for optimization is to increase expression levels of tesB through selection of appropriate promoters and ribosome binding sites. In some embodiments this may include the selection of high-copy number plasmids, or low or medium-copy number plasmids. The step of transcription termination can also be targeted for regulation of gene expression, through the introduction or elimination of structures such as stem-loops.

In some embodiments it may be advantageous to use a cell that has been optimized for production of one or more hydroxyacids. For example it may be optimal to mutate one or more components of the levulinate metabolism pathway to eliminate competing pathways and produce more of the one or more hydroxyacids. In some embodiments, screening for mutations that lead to enhanced production of one or more hydroxyacids may be conducted through a random mutagenesis screen, or through screening of known mutations. In some embodiments shotgun cloning of genomic fragments could be used to identify genomic regions that lead to an increase in production of one or more hydroxyacids, through screening cells or organisms that have these fragments for increased production of one or more hydroxyacids. In some cases one or more mutations may be combined in the same cell or organism.

Optimization of protein expression may also require in some embodiments that a gene encoding an enzyme be modified before being introduced into a cell such as through codon optimization for expression in a bacterial cell. Codon usages for a variety of organisms can be accessed in the Codon Usage Database (http://www.kazusa.or.jp/codon/).

In some embodiments protein engineering can be used to optimize expression or activity of an enzyme such as TesB. In certain embodiments a protein engineering approach could include determining the 3D structure of an enzyme or constructing a 3D homology model for the enzyme based on the structure of a related protein. Based on 3D models, mutations in an enzyme can be constructed and incorporated into a cell or organism, which could then be screened for an increased production of one or more hydroxyacids. In some embodiments hydroxyacid production in a cell could be increased through manipulation of enzymes that act in the same pathway as the enzymes associated with the invention. For example in some embodiments it may be advantageous to increase expression of an enzyme or other factor that acts upstream of a target enzyme such as tesB. This could be achieved by over-expressing the upstream factor using any standard method.

### EXAMPLES

### Example 1

### High-Titer Production of Monomeric Hydroxyvalerates from Levulinic Acid in Pseudomonas putida

Hydroxyacids represent an important class of compounds that see application in the production of polyesters, biodegradable plastics and antibiotics, and that serve as useful chiral synthetic building blocks for other fine chemicals and pharmaceuticals. It was reported that Pseudomonas putida accumulates PHA copolymers containing 3HV and 4HV when fed levulinic acid (Gorenflo, 2001). Levulinic acid is an inexpensive ketoacid that can be readily and renewably produced by treating wheat straw (Chang, 2007), corn starch (Cha, 2002), cellulose (Hayes, 2006) and other agricultural feedstocks with dilute acid at modestly elevated temperatures and pressures. In this study, a bioprocess for the production of monomeric 3HV and 4HV from levulinic acid in P. putida shake flask cultures was developed and optimized. Two strains of P. putida were tested: a commercially available strain (KT2440) and a PHA synthase knockout strain (GPp 104; Huisman, 1991). Two enzyme systems were also examined for removing CoA acyl carriers from intracellular hydroxyacids: the ptb/buk system and tesB. Once a suitable strain and enzyme system was found, the process was optimized at the shake flask scale in minimal and rich media for the high-titer production of both 4HV and 3HV.

The titer of 4HV and 3HV in shake flask cultures reached 10.84 g L⁻¹ and 4.71 g L⁻¹ respectively when P. putida tesB was cultured at 30° C. for 93 hours in LB medium, periodically supplemented with glucose and levulinate. The molar yields for the production of 4HV and 3HV from levulinate in these cultures reached 23.2% and 10.1% respectively. Additionally, 4HV and 3HV could be produced in shake flask culture in M9 minimal media up to titers of 2.04 g L⁻¹ and 1.43 g L⁻¹, respectively, with molar yields of 12.7% and 8.8% from levulinate.

### Materials and Methods

### Strains and Plasmids

Pseudomonas putida KT2440 (ATCC 47054; American Type Culture Collection, Manassas, Va., USA) and GPp104 (Huisman, 1991) were used to produce hydroxyvalerate monomers from levulinic acid. GPp104 is a polyhydroxyalkanoate (PHA) synthase deficient mutant of P. putida KT2442. Escherichia coli thioesterase II (tesB) was amplified from the E. coli K12 MG1655 (ATCC 47076) genome by PCR. The primers used were purchased from Sigma-Genosys (St. Louis, Mo., USA) and were as follows: 5′-GTCGACTTAATTGTGATTACGCATC-3′ (SEQ ID NO:1) and 5′-GAATTCTACTGGAGAGTTATATGAGTCAGG-3′ (SEQ ID NO:2). HotStar HiFidelity DNA polymerase was purchased from Qiagen (Valencia, Calif., USA) and used according to the manufacturer's instructions. The tesB gene was first cloned into the pGEM-T Easy vector (Promega, Madison, Wis., USA) to produce the plasmid pGEM-tesB. pGEM-tesB was then digested with SalI and EcoRI and cloned into a similarly digested broad-host-range expression vector pRK415 (Keen, 1988) to produce the plasmid pRK415-tesB. Molecular biology manipulations were performed using standard cloning protocols (Sambrook, 2001).

The phosphotransbutyryrase (ptb) and butyrate kinase (buk) genes were amplified as an operon by PCR from the genomic DNA of Clostridium acetobutylicum (ATCC 824) using the primers 5′-GAATTCACCAGTGATTAAGAGTTTTAATG-3′ (SEQ ID NO:3) and 5′-GTCGACGGTACTGGTTATATTATATTATTTATG-3′ (SEQ ID NO:4). These two genes were cloned into the pGEM-T Easy vector to yield the plasmid pGEM-ptb/buk. pGEM-ptb/buk was then digested with PstI and EcoRI and cloned into similarly digested pRK415 to produce the plasmid pRK415-ptb/buk.

### Chemicals and Growth Media

Dehydrated LB (Miller formulation) broth was purchased from BD Biosciences (San Jose, Calif., USA) and D-glucose was purchased from Mallinckrodt Chemicals (Phillipsburg, N.J., USA). M9 minimal medium was prepared as described elsewhere (Sambrook, 2001). LB and M9 media were autoclaved prior to use, while D-glucose was prepared as a 20% (w/v) stock solution and sterile filtered. Levulinic acid was purchased from Acros Organics (Morris Plains, N.J., USA) and was neutralized to a pH of 7.0 with 10N NaOH and sterile-filtered prior to use. ATCC Trace Mineral Supplement (ATCC MD-TMS) was added to M9 cultures where indicated. 3-Hydroxyvalerate was purchased from Epsilon Chimie (Brest, FRANCE) and was used as an HPLC standard. 4-Hydroxyvalerate was made by saponification of γ-valerolactone purchased from Alfa Aesar (Ward Hill, Mass., USA) and was used as an HPLC standard.

Culturing of P. putida

50 mL of M9 or LB media was added to a 250 mL shake flask. The media was supplemented with 10 μg mL⁻¹ tetracycline for plasmid maintenance, 1.0 mM IPTG for induction of gene expression, and various levulinic acid concentrations. Some M9 cultures were given a 1000× dilution of ATCC Trace Mineral Supplement as indicated. The inocula for all cultures was prepared from P. putida KT2440 or GPp104 grown overnight from frozen stock in LB at 30° C., centrifuged at 2,000×g and 4° C. for 5 min, and washed and resuspended in 0.9% (w/v) sodium chloride. All experimental cultures were inoculated with this resuspension to an initial optical density at 600 nm (OD₆₀₀) of 0.05 and were incubated at 30 or 32° C. with shaking at 250 rpm. Samples were periodically withdrawn from these cultures for OD₆₀₀ and HPLC analysis.

### Analytics

Culture cell density was monitored by measuring optical density at 600 nm (OD₆₀₀) on a Beckman Coulter DU800 UV/V is spectrophotometer. Optical density readings of cell concentration were correlated to dry cell weight (DCW) per unit volume by measuring the OD₆₀₀ of several P. putida KT2440 or GPp104 cultures, filtering a known culture volume through a pre-weighed Whatman 0.45 μm cellulose acetate filter, and drying the retained cells for several days in an oven. A calibration curve for both KT2440 and GPp 104 was constructed to convert OD₆₀₀ values to g DCW-L⁻¹ and conversion factors of 0.4234 g DCW-L⁻¹ OD₆₀₀⁻¹ and 0.5284 g DCW-L⁻¹ OD₆₀₀⁻¹ were found for KT2440 and GPp104, respectively.

High-performance liquid chromatography (HPLC) samples were prepared by centrifuging 1 mL of culture at 14,000×g for 10 min at room temperature and withdrawing the supernatant for analysis. HPLC samples were analyzed on an Agilent 1100 Series HPLC fitted with an Agilent ZORBAX SB-Aq reverse phase column (4.6×150 mm, 3.5 μm). The column temperature was maintained at 65° C. Levulinic acid and hydroxyvalerate concentrations were measured with a refractive index detector. The mobile phase was an aqueous solution of 25 mM ammonium formate at a pH of 2.0. The flowrate through the column was as follows: 0.250 mL min⁻¹ for the first 20 min, a linear increase in the flowrate from 0.250 to 1.000 mL min⁻¹ over 1 min, 1.000 mL min⁻¹ for the next 14 min, a linear decrease in the flowrate from 1.000 to 0.250 mL min⁻¹ over 1 min, and 0.250 mL min⁻¹ for an additional 14 min. Levulinic acid, 3-hydroxyvalerate, and 4-hydroxyvalerate were used as HPLC standards.

### Results

Effect of ptb/buk, tesB and PHA Synthase Deficiency on Hydroxyvalerate Titer

Recombinant P. putida KT2440 or GPp104 harboring the plasmid pRK415 (empty plasmid control), pRK415-ptb/buk, or pRK415-tesB was grown in 50 mL LB shake flask culture at 30° C. Levulinate was added in 50 mM aliquots at t=0, 6, 22, 29, and 49 hours. The resulting titers of 4HV and 3HV reached 4.08 g L⁻¹ and 1.11 g L⁻¹ respectively in the KT2440(pRK415-tesB) culture after 72 hours (Table 1). The molar yields of 4HV and 3HV from the levulinate consumed in this culture were 15.3% and 4.2%, respectively, at 72 hours.

The expression of tesB leads to a dramatic increase in both 4HV and 3HV concentration in the culture media while the ptb and buk genes do not improve either of the titers (Table 1). Furthermore, P. putida GPp104 in all cases had lower 4HV titers than KT2440 and did not produce detectable amounts of 3HV. GPp 104 performance was comparable with KT2440 for 4HV production when the 4HV titer is normalized with respect to cell density, indicating that both strains produce the same amount of 4HV on a per cell basis. The fact that both strains had similar normalized 4HV titers suggests that PHA accumulation is not competing significantly with 4HV production in KT2440; the differences in titer between KT2440 and GPp104 are most likely due to differences in stationary phase cell density rather than the presence or absence of PHA synthase activity. These results indicated that KT2440 was a superior strain for the production of hydroxyvalerates, and consequently all future investigations were done with KT2440.

### Production of Hydroxyvalerate in LB Medium

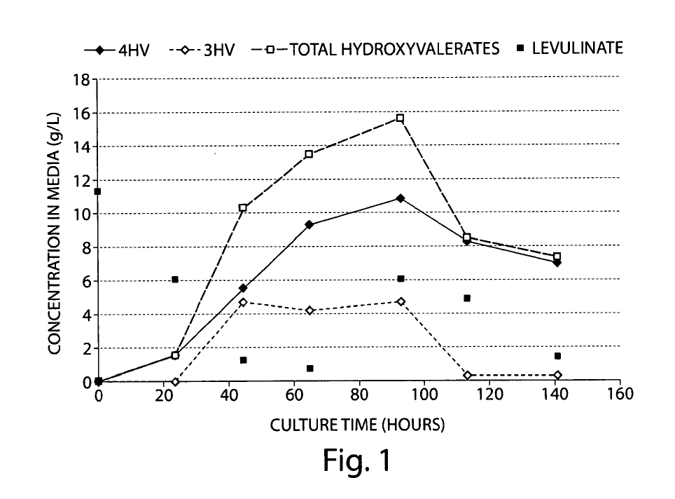

Recombinant P. putida KT2440 harboring the plasmid pRK415 or pRK415-tesB was grown in 50 mL LB shake flask cultures at 30° C. Where indicated, cultures were supplemented with either 0.4% (w/v) glucose or an initial 0.4% glucose followed by an additional 0.4% glucose at t=24, 45 and 65 hours. Levulinate was fed to the cultures as follows: 100 mM levulinate at t=0, 24, 45 hours and 150 mM levulinate at t=65 hours. The KT2440(pRK415-tesB) culture yielded a 4HV titer of 10.84 g L⁻¹ and a 3HV titer of 4.71 g L⁻¹ after 93 hours (FIG. 1). The respective molar yields of 4HV and 3HV from the levulinate consumed in this culture were 23.2% and 10.1% (Table 2). The addition of glucose significantly improved both hydroxyvalerate titers as well as their yield from levulinate.

The time-course for hydroxyvalerate production for the KT2440 LB culture harboring pRK415-tesB and fed 0.4% glucose periodically is shown in FIG. 1. This time-course is typical for KT2440 expressing tesB in LB. The concentration of 4HV and 3HV rises over time as long as levulinate is present in the culture media. After 3-4 days, the hydroxyvalerate titers plateau and then begin to decline, even in the presence of levulinate. The 3HV in the media is rapidly consumed while the 4HV titer decreases more slowly.

### Production of Hydroxyvalerates in M9 Minimal Medium

Recombinant P. putida KT2440 harboring the plasmid pRK415-tesB was grown in M9 minimal medium in 50 mL shake flasks under a variety of conditions. Specifically, hydroxyvalerate production was tested in M9 with or without ATCC trace mineral supplement, with 0.4% (w/v) glucose, and in cultures incubated at 30° C. or 32° C. Levulinate was added to these cultures in portions of 25, 25, 50, 50, and 50 mM at t=0, 23, 45, 67, and 93 hours respectively. The results of this experiment after 93 hours for the M9 culture grown at 32° C. and 65 hours for all other cultures are shown in Table 3.

### Effect of Temperature on Hydroxyvalerate Production

In the course of experimentation it was discovered that temperature influences both the overall hydroxyvalerate titer and the ratio of 4HV to 3HV produced from levulinate in P. putida. To test this phenomenon explicitly, recombinant P. putida KT2440 harboring the plasmid pRK415-tesB was grown in LB media in 50 mL shake flasks at 30° C. and 32° C. Levulinate was added to these cultures in portions of 100, 150, 150, and 150 mM at t=0, 17, 41, and 65 hours respectively. In some instances, cultures were supplemented with either 0.4% (w/v) glucose or an initial 0.4% glucose followed by an additional 0.4% glucose at t=17, 41, and 65 hours.

Production of 3HV from 4HV

To gain insight into the pathway by which 4HV and 3HV are produced from levulinate, an experiment was carried out to produce 3HV directly from 4HV. P. putida KT2440 harboring the plasmid pRK415-tesB was grown in LB supplemented with 0.4% (w/v) glucose, 1 mM IPTG, and 4HV. The 4HV feedstock was prepared by saponification of commercially purchased γ-valerolactone.

### Discussion

Herein, it is demonstrated that high concentrations of 4HV and 3HV can be made from levulinate in recombinant P. putida KT2440 by overexpressing the tesB gene. By carefully optimizing the culture and feeding conditions, titers of 4HV and 3HV in excess of 10 g L⁻¹ and 4 g L⁻¹ respectively have been achieved at the shake flask scale.

Hydroxyvalerate production in M9 minimal medium in addition to LB was further investigated. While the titers of both 4HV and 3HV are expectedly lower in M9 relative to LB, both 4HV and 3HV can still be produced at the g L⁻¹ scale in minimal media shake flasks. Interestingly, growth in M9 encourages the higher accumulation of 3HV relative to 4HV, while growth in LB yields a higher ratio of 41HV to 3HV (Tables 2 and 3). The addition of trace mineral supplement to the M9 cultures also increases the amount of 3HV relative to 4HV (Table 3). Temperature also appears to influence the hydroxyvalerate ratio, with higher temperatures favoring 4HV production over 3HV. P. putida lacking the tesB gene have very high 4HV:3HV ratios as they produce little or no 3HV, but the total hydroxyvalerate yield from these cultures is several fold lower than those expressing tesB (Table 1).

In the course of experimentation, it was noticed that when P. putida ran out of levulinate, it began metabolizing 3HV. Once 3HV was depleted, the cells would begin to catabolize 4HV. This suggests that levulinate is metabolized by P. putida via fatty acid oxidation. The appearance of 4HV in the culture media when P. putida grows on levulinate implies that a reductase in P. putida must be capable of reducing the keto group in levulinate to a hydroxyl group, forming the 4HV. Without wishing to be bound by any theory, the 3HV observed in this study my be produced from 4HV by β-γ dehydration of the 4HV hydroxyl group and the subsequent hydration of the alkene bond at the β-position. The 3HV, a β-oxidized fatty acid, can then be degraded to propionyl-CoA and acetyl-CoA, both of which eventually feed into the tricarboxylic acid cycle of the cell. In this study, 4HV titers were consistently higher than the 3HV titers, suggesting that conversion of 4HV into 3HV is slower than the reduction of levulinate into 4HV. The detection of 3HV at any level further suggests that the conversion of 4HV to 3HV is faster than the catabolism of 3HV into citric acid cycle intermediates. If the specific enzymes responsible for the steps in the levulinate metabolism pathway are found, the pathway could likely be made to produce even higher titers of 3HV or 4HV from levulinate through more precise metabolic engineering such as the elimination of competing pathways. Additionally, discovery of the levulinate pathway enzymes would allow the reconstruction of the pathway in other organisms like Escherichia coli.

Given that total hydroxyvalerate titers in excess of 15 g L⁻¹ could be made in 50 mL shake flasks, one future study of immediate interest is hydroxyvalerate production in larger stirred tank reactors. Large-scale production in a better aerated reaction vessel should afford even higher titers of 4HV and 3HV with improved economic margins. Another future study of interest is the production of other hydroxyacids with tesB in P. putida. In particular, it is known that supplying P. putida with oleic acid results in the accumulation of a PHA containing 3-hydroxyalkanoic acids from six to fourteen carbon atoms in length (De Roo, 2002). Given that tesB is known to accept longer-chain fatty acid CoA thioesters such as decanoyl-CoA as substrates (Naggert, 1991), it is likely that tesB will be able to liberate these C₆-C₁₄ 3-hydroxyacids from their intracellular CoA carriers prior to their incorporation as a

PHA. This would result in the accumulation of these novel, longer-chain hydroxyacids in the culture media.

In conclusion, a method was developed for producing high titers of the high-value hydroxyacids 4HV and 3HV from the renewable and inexpensive substrate levulinate in P. putida KT2440 at the shake flask scale by overexpressing the tesB gene. Hydroxyvalerate titers of several grams per liter were achieved in both rich and minimal media. Optimization of culture conditions such as temperature, feed profile, and media yielded increased hydroxyvalerate titers as well as some control over the relative amounts of 4HV and 3HV produced.

TABLE 1
Table 1: Hydroxyvalerate accumulation in shake flask culture media after 72 hours by recombinant
P. putida KT2440 or GPp104 grown at 30° C. in LB and harboring different plasmids.

| | | | KT2440 | GPp104 | KT2440 | GPp104 |
|---|---|---|---|---|---|---|
| | KT2440 | GPp104 | ptb + buk | ptb + buk | tesB | tesB |
| Cell Density (g DCW L⁻¹): | 4.85 | 1.55 | 2.61 | 2.32 | 2.51 | 1.44 |
| Total Levulinate Consumed (g L⁻¹): | 28.8 | 9.5 | 25.3 | 13.6 | 27.1 | 7.2 |
| 4HV Titer (g L⁻¹): | 1.50 | 0.42 | 0.53 | 0.53 | 4.08 | 1.58 |
| 3HV Titer (g L⁻¹): | 0.51 | 0.00 | 0.56 | 0.00 | 1.11 | 0.00 |
| Normalized 4HV Titer (g L⁻¹ OD₆₀₀⁻¹): | 0.130 | 0.144 | 0.086 | 0.121 | 0.689 | 0.581 |
| Normalized 3HV Titer (g L⁻¹ OD₆₀₀⁻¹): | 0.045 | 0.000 | 0.090 | 0.000 | 0.187 | 0.000 |
| 4HV Molar Yield (mM 4HV mM Lev⁻¹): | 5.3% | 4.5% | 2.1% | 4.0% | 15.3% | 22.3% |
| 3HV Molar Yield (mM 4HV mM Lev⁻¹): | 1.8% | 0.0% | 2.2% | 0.0% | 4.2% | 0.0% |

Levulinate was fed to this cultures in 50 mM increments at t = 0, 6, 22, 29, and 49 hours.

TABLE 2
Table 2: Hydroxyvalerate accumulation in shake flask culture media after 93 hours by recombinant
P. putida KT2440 grown at 30° C. in LB and harboring either pRK415 or pRK415-tesB.

| | | | | LB + 0.4% | LB + 0.4% | LB + 0.4% |
|---|---|---|---|---|---|---|
| | | LB | LB + 0.4% | Glucose | Glucose per | Glucose per |
| | LB | pRK415- | Glucose | pRK415- | 24 hrs | 24 hrs |
| | pRK415 | tesB | pRK415 | tesB | pRK415 | pRK415-tesB |
| Cell Density (g DCW L⁻¹): | 4.70 | 3.30 | 2.96 | 3.16 | 3.16 | 3.57 |
| Total Levulinate Consumed (g L⁻¹): | 39.0 | 47.3 | 35.9 | 48.8 | 37.4 | 45.9 |
| 4HV Titer (g L⁻¹): | 1.07 | 4.24 | 1.09 | 5.17 | 1.06 | 10.84 |
| 3HV Titer (g L⁻¹): | 0.00 | 1.56 | 0.00 | 1.73 | 0.00 | 4.71 |
| 4HV Molar Yield (mM 4HV mM Lev⁻¹): | 2.7% | 8.8% | 3.0% | 10.4% | 2.8% | 23.2% |
| 3HV Molar Yield (mM 4HV mM Lev⁻¹): | 0.0% | 3.3% | 0.0% | 3.5% | 0.0% | 10.1% |

Glucose was added to cultures where indicated either as a 0.4% (w/v) bolus at the start of the culture or as a 0.4% (w/v) bolus periodically approximately every 24 hours. Levulinate was fed to the cultures as follows: 100 mM levulinate at t = 0, 24, 45 hours and 150 mM levulinate at t = 65 hours.

TABLE 3
Table 3: Hydroxyvalerate accumulation in shake flask culture media by recombinant
P. putida KT2440 harboring pRK415-tesB grown at 30° C. or 32° C. in M9 minimal media.

| | M9 | M9 | M9 | M9 |
|---|---|---|---|---|
| | 30° C. | 30° C. + TM | 32° C. | 32° C. + TM |
| | (t = 65 Hrs) | (t = 65 Hrs) | (t = 93 Hrs) | (t = 65 Hrs) |
| Cell Density (g DCW L⁻¹): | 0.43 | 2.3 | 1.17 | 1.6 |
| Total Levulinate Consumed (g L⁻¹): | 6.8 | 11.5 | 15.9 | 11.2 |
| 4HV Titer (g L⁻¹): | 0.51 | 0.16 | 2.04 | 0.98 |
| 3HV Titer (g L⁻¹): | 0 | 1.55 | 1.43 | 1.62 |
| 4HV Molar Yield | 7.3% | 1.4% | 12.7% | 8.6% |
| (mM 4HV mM Lev⁻¹): | | | | |
| 3HV Molar Yield | 0.0% | 13.3% | 8.8% | 14.2% |
| (mM 4HV mM Lev⁻¹): | | | | |

An initial 0.4% (w/v) of glucose was given to each culture. ATCC trace mineral supplement (TM) was supplemented into the cultures where indicated. Levulinate was added to these cultures in portions of 25, 25, 50, 50, and 50 mM at t = 0, 23, 45, 67, and 93 hours respectively.

### REFERENCES

Cha J Y and Hanna M A, “Levulinic acid production based on extrusion and pressurized batch reaction,” Ind. Crop. Prod., 2002, 16:109-118.
Chang C, Cen P and Ma X, “Levulinic acid production from wheat straw,” Bioresource Technol., 2007, 98:1448-1453.
Chen G Q and Wu Q, “Microbial production and applications of chiral hydroxyalkanoates,” Appl. Microbiol. Biotechnol., 2005, 67:592-599.
Chiba T and Nakai T A, “Synthetic Approach to (1)-thienamycin from methyl (R)-(2)-3-hydroxybutanoate. A new entry to (3R,4R)-3-[(R)-1-hydroxyethyl-4-acetoxy-2-azetidinone,” Chem. Lett., 1985, 5:651-654.
De Roo G, Kellerhals M B, Ren Q, Witholt B and Kessler B, “Production of chiral R-3-hydroxyalkanoic acids and R-3-hydroxyalkanoic acid methylesters via hydrolytic degradation of polyhydroxyalkanoate synthesized by Psuedomonads,” Biotechnol. Bioeng., 2002, 77:717-722.

Gao H J, Wu Q and Chen G Q, “Enhanced production of D-(−)-3-hydroxybutyric acid by recombinant Escherichia coli,” FEMS Microbiol. Lett., 2002, 213:59-65.
Gorenflo V, Schmack G, Vogel R and Steinbüchel A, “Development of a Process for the Biotechnological Large-Scale Production of 4-Hydroxyvalerate-Containing Polyesters and

Characterization of Their Physical and Mechanical Properties,” Biomacromolecules, 2001, 2:45-57.
Hayes D J, Fitzpatrick S, Hayes M H B and Ross J R H, “The Biofine process—production of levulinic acid, furfural, and formic acid from lignocellulosic feedstocks.” In: Kamm B, Gruber P R, Kamm M (eds) Biorefineries—Industrial Processes and Products. 2006, Wiley-VCH, Weinheim, pp. 139-164.

Hazer B and Steinbüchel A, “Increased diversification of polyhydroxyalkanoates by modification reactions for industrial and medical applications,” Appl. Microbiol. Biotechnol., 2007, 74:1-12.

Huisman G W, Wonink E, Meima R, Kazemier B, Terpstra P, and Witholt, B, “Metabolism of Poly(3-hydroxyalkanoates) (PHAs) by Pseudomonas oleovorans,” J. Biol. Chem., 1991, 266:2191-2198.
Jaipuri F A, Jofre M F, Schwarz K A and Pohl N L, “Microwave-assisted cleavage of Weinreb amide for carboxylate protection in the synthesis of a (R)-3-hydroxyalkanoic acid,” Tetrahed. Lett., 2004, 45:4149-4152.
Keen N T, Tamaki S, Kobayashi D and Trollinger D, “Improved broad-host-range plasmids for DNA cloning in Gram-negative bacteria,” Gene, 1988, 70:191-197.
Lee S Y, Lee Y and Wang F, “Chiral Compounds from Bacterial Polyesters: Sugars to Plastics to Fine Chemicals,” Biotechnol Bioeng, 1999, 65:363-368.
Lee S Y, Park S H, Lee Y and Lee S H (2002), “Production of chiral and other valuable compounds from microbial polyesters.” In: Doi Y, Steinbüchel A (eds) Biopolymers, polyesters III. Wiley-VCH, Weinheim, pp. 375-387.
Lenz R W and Marchessault R H, “Bacterial polyesters: biosynthesis, biodegradable plastics and biotechnology,” Biomacromolecules, 2005, 6:1-8.

Liu Q, Ouyang S P, Chung A, Wu Q and Chen G Q, “Microbial production of R-3-hydroxybutyric acid by recombinant E. coli harboring genes of phbA, phbB, and tesB,” Appl. Microbiol. Biotechnol., 2007, 76:811-818.
Liu S J and Steinbüchel A, “A novel genetically engineered pathway for synthesis of poly(hydroxyalkanoic acids) in Escherichia coli,” Appl Environ Microbiol, 2000a, 66:739-743.
Liu S J and Steinbüchel A, “Exploitation of butyrate kinase and phosphotransbutyrylase from Clostridium acetobutylicum for the in vitro biosynthesis of poly(hydroxyalkanoic acid),” Appl Microbiol Biotechnol, 2000b, 53:545-552.
Naggert J, Narasimhan M L, DeVeaux L, Cho H, Randhawa Z I, Cronan J E, Green B N and Smith S, “Cloning, sequencing, and characterization of Escherichia coli thioesterase II,” J. Biol. Chem., 1991, 266:11044-11050.
Park S H, Lee S H and Lee S Y, “Preparation of optically active beta-amino acids from microbial polyester polyhydroxyalkanoates,” J. Chem. Res. Synop., 2001, 11:498-499. Park S J and Lee, S Y, “Biosynthesis of poly(3-hydroxybutyrate-co-3-hydroxyalkanoates) by metabolically engineered Escherichia coli strains,” Appl. Biochem. Biotechnol., 2004, 113-116:335-346.
Park S J, Park J P and Lee S Y, “Metabolic engineering of Escherichia coli for the production of medium-chain-length polyhydroxyalkanoates rich in specific monomers,” FEMS Microbiol. Lett., 2002, 214:217-222.
Ren Q, Ruth, K, Thony-Meyer L and Zinn M, “Process Engineering for Production of Chiral Hydroxycarboxylic Acids from Bacterial Polyhydroxyalkanoates,” Macromol. Rapid Commun., 2007, 28:2131-2136.
Sambrook J and Russell D W, “Molecular Cloning: a Laboratory Manual,” 3^{rd} Ed. 2001, Cold Spring Harbor Laboratory, Cold Spring Harbor, N.Y.
Seebach D, Albert M, Arvidsson P I, Rueping M and Schreiber J V, “From the biopolymer PHB to biological investigations of unnatural beta- and gamma-peptides,” Chimia, 2001, 55:345-353.
Seebach D, Beck A K, Breitschuh R and Job K, “Direct degradation of the biopolymer [(R)-3-hydroxybutyric acid] to (R)-3-hydroxybutanoic acid and its methyl ester,” Org. Synth., 1998, 71:39-43.
Tasaki O, Hiraide A, Shiozaki T, Yamamura H, Ninomiya N and Sugimoto H, “The dimer and trimer of 3-hydroxybutyrate oligomer as a precursor of ketone bodies for nutritional care,” J. Parenter Enteral Nutr, 1999, 23:321-325.
Wang F and Lee S Y, “Poly(3-hydroxybutyrate) production with high productivity and high polymer content by a fed-batch culture of Alcaligenes latus under nitrogen limitation,” Appl. Environ. Microbiol., 1997, 63:3703-3706.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A cell that produces one or more hydroxyacids, wherein the cell recombinantly expresses tesB, optionally wherein the tesB gene is an E. coli gene.

2. The cell of claim 1 wherein the cell converts levulinic acid to the one or more hydroxyacids.

3. The cell of claim 1 wherein the hydroxyacid comprises 3-hydroxyvalerate (3HV) and/or 4-hydroxyvalerate (4HV).

4. The cell of claim 1 wherein the cell is a bacterial cell, a fungal cell (including a yeast cell), a plant cell, an insect cell or an animal cell.

5. The cell of claim 4 wherein the cell is a bacterial cell.

6. The cell of claim 5 wherein the cell is a Pseudomonas cell, optionally a Pseudomonas putida cell, optionally a Pseudomonas putida KT2440 strain cell.

7. (canceled)

8. (canceled)

9. The cell of claim 1 wherein the tesB gene is expressed from a plasmid or is integrated into the genome of the cell.

10. (canceled)

11. (canceled)

12. A method for producing one or more hydroxyacids, the method comprising culturing the cell of claim 1 to produce one or more hydroxyacids, optionally further comprising recovering the one or more hydroxyacids from the cells.

13. (canceled)

14. The method of claim 12 wherein one or more of the hydroxyacids produced is 3-hydroxyvalerate (3HV), and wherein the titer of 3-hydroxyvalerate (3HV) produced is at least 1 g L⁻¹ in minimal media, and at least 4 g L⁻¹ in rich media.

15. The method of claim 12 wherein one or more of the hydroxyacids produced is 4-hydroxyvalerate (4HV), and wherein the titer of 4-hydroxyvalerate (4HV) produced is at least 1.5 g L⁻¹ in minimal media, and at least 9 g L⁻¹ in rich media.

16. A method for producing a cell that has increased hydroxyacid production comprising recombinantly expressing tesB in the cell, optionally wherein the tesB gene is an E. coli gene.

17. The method of claim 16 wherein the cell is provided with levulinic acid, optionally wherein the hydroxyacid comprises 3-hydroxyvalerate (3HV) and/or 4-hydroxyvalerate (4HV).

18. (canceled)

19. The method of claim 1 wherein the cell is a bacterial cell, a fungal cell (including a yeast cell), a plant cell, an insect cell or an animal cell.

20. The method of claim 19 wherein the cell is a bacterial cell.

21. The method of claim 20 wherein the cell is a Pseudomonas cell, optionally a Pseudomonas putida cell, optionally a Pseudomonas putida KT2440 strain cell.

22. (canceled)

23. (canceled)

24. The method of claim 16 wherein the tesB gene is expressed on a plasmid or is integrated into the genome of the cell.

25. (canceled)

26. (canceled)

27. A method for producing one or more hydroxyacids, the method comprising producing a cell according to the method of claim 16, culturing a population of said cells, and collecting one or more hydroxyacids from the population of cells.

28. The method of claim 16 wherein one or more of the hydroxyacids produced is 3-hydroxyvalerate (3HV), and wherein the titer of 3-hydroxyvalerate (3HV) produced is at least 1 g L⁻¹ in minimal media, and at least 4 g L⁻¹ in rich media.

29. The method of claim 16 wherein one or more of the hydroxyacids produced is 4-hydroxyvalerate (4HV), and wherein the titer of 4-hydroxyvalerate (4HV) produced is at least 1.5 g L⁻¹ on minimal media, and at least 9 g L⁻¹ on rich media.

30.-40. (canceled)


SEQUENCE LISTING (4 sequences)

SEQ ID NO: 1 - DNA, 25 bp, Artificial Sequence
  synthetic oligonucleotide
gtcgacttaattgtgattacgcatc

SEQ ID NO: 2 - DNA, 30 bp, Artificial Sequence
  synthetic oligonucleotide
gaattctactggagagttatatgagtcagg

SEQ ID NO: 3 - DNA, 29 bp, Artificial Sequence
  synthetic oligonucleotide
gaattcaccagtgattaagagttttaatg

SEQ ID NO: 4 - DNA, 33 bp, Artificial Sequence
  synthetic oligonucleotide
gtcgacggtactggttatattatattatttatg